(19) United States
(12) Patent Application Publication
(10) Pub. No.: US 2008/0287933 A1
(43) Pub. Date: Nov. 20, 2008

(54) Multifiber Instrument for Contact Laser Surgery

(76) Inventor: Roberto Pini, Firenze (IT)

Correspondence Address: MCGLEW & TUTTLE, PC, P.O. BOX 9227, SCARBOROUGH STATION, SCARBOROUGH, NY, 10510-9227, US

(21) Appl. No.: 12/067,492

(22) PCT Filed: Sep. 19, 2006
(86) PCT No.: PCT/IT2006/000663

§ 371 Date: Jul. 3, 2008

(30) Foreign Application Priority Data
Sep. 22, 2005 (IT) FI2005A0000196

(51) Int. Cl. A61B 18/22

(52) U.S. Cl. 606/10; 606/16

(57) ABSTRACT

The surgical instrument comprises a handpiece (1), associated with which is a tip (5) forming a wave guide and terminating with an operating end, intended to act on the tissues and from which a laser radiation is emitted and which receives said laser radiation from fiber optic means (4). The tip is implemented to collect the laser emission coming from a plurality of optical fibers couplable with said tip at a coupling face and to convey said laser emissions towards said operating end.

### TECHNICAL FIELD

The present invention relates to a surgical instrument for laser surgery, of the type comprising a handpiece terminating with a tip forming an operating end, from which laser energy, coming from optical fibers, is emitted to perform cutting, abrasion or other operations on biological tissues.

### PRIOR ART

As is known, lasers are currently used in surgery as instruments to induce cutting, coagulation, vaporization, ablation or photodestruction of various types of biological tissue. Particularly important uses are those in which laser radiation is employed for resection or eradication of tumors in various areas of the human body. Very often use of the laser provides substantial advantages compared to conventional surgical instruments, as the cut is implemented in a precise manner, comparable to one made with conventional scalpels, and with the development of a coagulative and hemostatic action, better controlled and more localized than one implemented with the “electric scalpel” (bipolar probe). In these applications in addition to CO₂ lasers, one of the most often utilized lasers is the Neodymium:YAG (abbreviation Nd:YAG, wavelength 1064 nm) with continuous emission, which has good transmission and diffusion in tissues, with penetration depths which can reach several millimeters, depending on the type of tissue. In order to localize the action of this laser to lower penetration depths, to better control the cutting and coagulative action, the Nd:YAG is typically operated in “contact” mode, i.e. using a fiber optic handpiece terminating in a usually tapered sapphire tip, which concentrates the emission of radiation on said tip, and therefore only acts when the tip comes into contact with the tissue to be cut.

As a practical example of surgical use of this technique, surgical laser resection of meningiomas (benign tumors which represent 15% of brain tumors and 25% of spinal tumors) in neurosurgery can be cited, in which an Nd:YAG laser in “contact” mode has proved capable of implementing total surgical removal of the tumor, with significant advantages compared to conventional non-laser techniques. The laser powers employed vary in these operations typically between 10 and 100 Watts continuous.

Diode lasers have recently been proposed as potential replacements for Nd:YAG surgical lasers, typically with emission in the 800-960 nm spectral region, which have a type of interaction with organic tissues (in terms of penetration depth and heat development) similar to continuous Nd:YAG lasers, but with unquestionable advantages from the technological viewpoint, such as: much smaller dimensions, greater wall-plug efficiency (ratio between laser power emitted and electrical power absorbed from the network), which consequently involves less energy consumption and a simplified cooling system, increased electromagnetic compatibility, which is a crucial aspect for use in the operating theater. On the other hand, the diode lasers with emission in the spectral region indicated above have limited costs only for powers in the range of 10 Watts, as higher powers require more complicated and costly technologies in order to overcome problems of overheating of the emitting couplings which also limit their useful life.

Examples of handpieces for laser surgical instruments are described in the U.S. Pat. Nos. 4,538,609, 4,627,435, 5,352,221 and 5,662,646.

In particular, U.S. Pat. No. 4,627,435 describes a handpiece with a tip, tapered or cylindrical in shape and beveled in the form of a wedge.

### OBJECTS AND SUMMARY OF THE INVENTION

An object of the present invention is to provide a surgical instrument for laser surgery that can utilize diode laser sources overcoming the limits in terms of power indicated above.

This and other objects and advantages, which will be apparent to those skilled in the art from the text hereunder, are obtained with the features of claim 1. The dependent claims indicate further possible advantageous and preferred features.

According to a different aspect, the invention also relates to a surgical instrument as defined in claim 12. Further features of the instrument according to the invention are indicated in the dependent claims.

The object of an improved embodiment of the invention is to provide a device that allows transmission to the tissue to be treated of various laser emissions at different wavelengths, which simultaneously induce different surgical and/or therapeutic effects.

For this purpose the multiple laser sources, which inject energy into the individual fibers terminating in the instrument according to the invention are designed to emit at different wavelengths.

This consequently offers a further advantage in the use of a multifiber handpiece according to the invention, represented by the possibility of superimposing in the same point of application to the tissue to be treated the emissions of lasers of various wavelengths, simultaneously obtaining different therapeutic effects. For example, it is known that hemoglobin has selective absorption in the green-yellow spectral region, with peaks around 540 and 580 nm (see, for example, S. Takami an M. D. Graham, IEEE Trans. Biomed. Eng., BME-26, 656-664, 1987). Therefore, the cutting action of a medium power laser in the near infrared could be advantageously combined in a single “contact” handpiece with that of a medium-low power green laser, employed to selectively induce hemostasis of the severed vessels.

In substance, according to the invention, the emission of various laser devices is collected through distinct optical fibers, so that, through a suitably designed coupling system, these fibers are connected to a tip made of suitable material, such as sapphire. In particular, the dimensions and shape of this tip are suitably designed to collect most of the laser emission of the optical fibers and convey it to the point of surgical treatment. This tip can have a truncated-cone or other shape. In the truncated-cone configuration, for example, the tip can be designed so that a substantial fraction (up to 90%) of the laser radiation entering through the larger base (input face) is guided towards the smaller base (output face). From this it is then emitted with high angular divergence, so that the surgical action is selectively localized in proximity of said output face, satisfying the requirements of surgical use in “contact” mode. This tip is advantageously interchangeable through a suitable connector. The final section of the optical fibers, the coupling system of these fibers with the tip and the connector of said tip are included in a suitably designed handpiece which allows easy manual operation by the surgeon, even under the surgical microscope.

### BRIEF DESCRIPTION OF THE DRAWINGS

Further features and advantages of the multifiber contact handpiece for surgical use, according to the present invention, will be more apparent from the description hereunder of an embodiment thereof, provided purely by way of a non-limiting example with reference to the accompanying drawings, in which:

FIG. 1 shows a principle diagram of the device according to the invention;

FIG. 2 schematically shows the overall view of a possible embodiment of the handpiece;

FIG. 3 shows the detail of a possible embodiment of the terminal part of the handpiece, comprising the tip, the relative connector and the system to connect the optical fibers to the tip.

FIG. 4 schematically shows a possible embodiment of the truncated-cone shaped tip, in which the optical paths of some beams are traced to show the guided focusing effect;

FIG. 5 shows the distribution of power delivered from the tip represented in the previous FIG. 4.

### DESCRIPTION OF THE PREFERRED EMBODIMENT

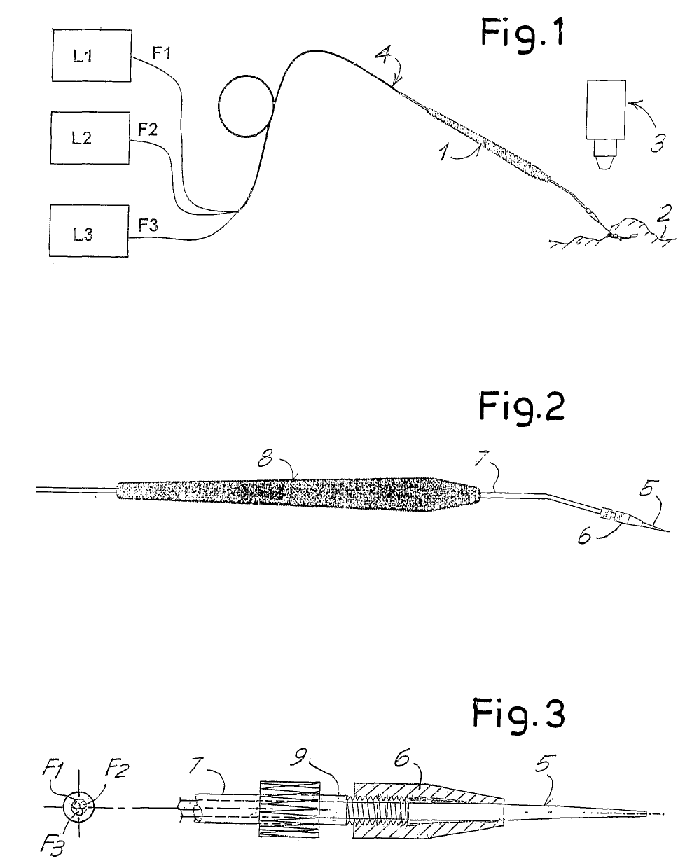

With reference to FIG. 1, which shows, by way of example, the embodiment of a handpiece with three optical fibers, (1) indicates the multifiber contact handpiece, (2) indicates the meningioma which is eradicated using the laser, (3) indicates the surgical microscope, (4) indicates a sheath containing the optical fibers, F1, F2 and F3 indicate the three optical fibers which are connected to three laser devices or sources, indicated with L1, L2 and L3, through SMA standard type connectors. By way of example, the three lasers can be represented respectively by two diode lasers with emission at 810 nm and maximum power of 10 Watts each (such as the Mod. SMARTY A-800 laser produced by El.En. spa, Italy), and by a diode-pumped and KDP doubled Nd:YAG laser with continuous emission at 532 nm and maximum power of 5 Watts (such as the SmartLite laser produced by El.En. spa).

This combination of wavelengths and laser powers allows the majority of meningiomas to be treated surgically, implementing simultaneously the laser cutting action through heat ablation and the selective hemostatic action on the blood vessels.

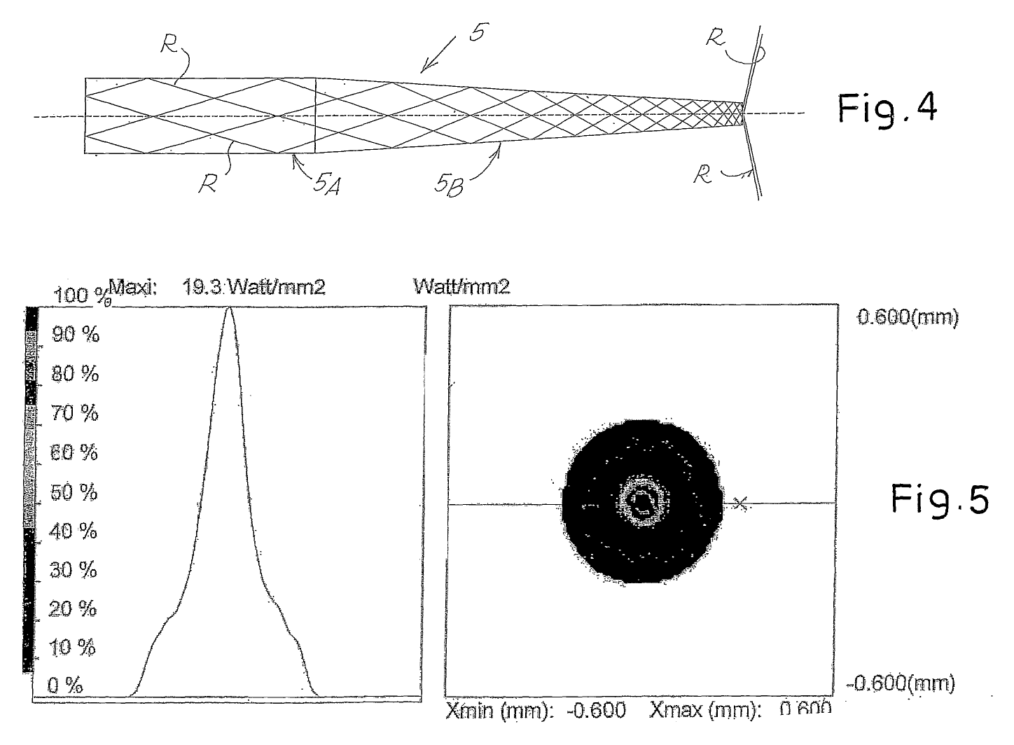

With reference to FIG. 2, which schematically shows the overall view of a possible embodiment of the handpiece, (5) indicates the tip, the shape and operating characteristics of which will be better described by means of FIGS. 4 and 5, (6) indicates the tip connector, (7) indicates the arm of the handpiece, made for example of stainless steel tube, (8) indicates the handgrip of the handpiece which can, for example, be made of plastic. It can be observed that a configuration of this type of the terminal part of the handpiece, equipped with a long, thin and slightly angled arm, at the end of which is the tip (5), allows practical use of the handpiece under the control of the surgical microscope, with minimum masking of the field of vision.

FIG. 3 shows the detail of a possible embodiment of the terminal part of the handpiece, where (5) indicates the tip, (6) indicates the tip connector, composed of a cap with a partially through hole, in the unthreaded part of which the tip (5) is constrained and the threaded part of which engages with the outer thread of a ferrule (9), against which the tip (5) is clamped and in which the terminal ends of the optical fibers, three in number in the example in the figure, indicated respectively with F1, F2 and F3 in the projection of the cross section of the ferrule, are contained and constrained.

The connector (6) allows easy interchangeability of the tip (5) in the case in which it requires to be replaced with another of different shape or to be cleaned, or because it is damaged during surgical procedures.

A possible embodiment of the tip (5) is shown in FIG. 4: it is composed of a cylindrical segment (5A), 1.65 mm in diameter and 8 mm in length and of a truncated-cone shaped segment (5B), which represents the terminal for “contact” laser emission, 15 mm in length and with circular output face 0.5 mm in diameter. The 1.65 mm diameter of the cylindrical segment, at the end of which the optical fibers (F1; F2; F3) interface, allows easy coupling, with minimum losses of emission, of three optical fibers with core diameter of 300 micron or less, including the overall dimensions of any sheaths thereof.

As known to those skilled in the art, there are beam tracing programs, such as the program Solstis by Optis (Toulon, France) which allow characterization, for a specific configuration of the tip (5), of the propagation modes of laser radiation for guided focusing and make it possible to obtain a wide angular divergence on the output face, fixed by the number and type of multiple fibers, to achieve high efficiency. An example of this tracing of beams is shown in, FIG. 4, where it can be seen that the beams (R), considered in the number of four to simplify the figure, propagate by total multiple reflections on the lateral surface of the tip to the output face, and from here are delivered with a wide angle of divergence, as required for “contact” surgical use.

Finally, considering a large number of beams, it is possible to calculate, with the same beam tracing programs mentioned above, the percentage of radiation effectively transmitted by the truncated cone shaped optical guide. For example, FIG. 5 shows the power distribution on the output face of the tip, with reference to the dimensions of the tip indicated in FIG. 4. In particular, the map on the right of FIG. 5 allows estimation for a tip of this type of propagation losses below 10%.

Variants and/or modifications can be made to the device forming the object of the present invention, without however departing from the protective scope of the invention as specified in the appended claims.

It is understood that the drawing only shows an example provided as a practical demonstration of the finding, which may vary in forms and arrangements without however departing from the scope of the concept on which the finding is based.

1. A surgical instrument for laser surgery, comprising:
a handpiece, associated with which is a tip forming a waveguide and terminating with an operating end, designed to act on the tissues and from which a laser radiation is emitted and which receives said laser radiation from a fiber optic device wherein said tip is designed to collect the laser emission coming from a plurality of optical fibers couplable with said tip at a coupling face and to convey said laser emissions towards said operating end.

2. Instrument as claimed in claim 1, wherein said operating end has a tapered shape.

3. Instrument as claimed in claim 2, wherein said operating end has a truncated-cone shape.

4. Instrument as claimed in claim 1, wherein said tip has an input portion, between said face for coupling with the optical fibers and said operating end, with a substantially constant cross section.

5. Instrument as claimed in claim 1, wherein said tip has a substantially cylindrical input portion.

6. Instrument as claimed in claim 1, further comprising a connector inside which said tip is fixed, said connector having means for coupling with a terminal, inside which first ends of a plurality of optical fibers are inserted, the second ends of which receive laser emission of respective laser radiation sources.

7. Instrument as claimed in claim 6, wherein said coupling means are screw means.

8. Instrument as claimed in claim 7, wherein said connector comprises an at least partly threaded through hole, fixed inside which is said tip, which projects from the connector from the side opposite to the coupling side between said connector and said terminal.

9. Instrument as claimed in claim 8, wherein said terminal constitutes the final part of a tubular element through which said fibers pass and integral with a handgrip of said handpiece.

10. Instrument as claimed in claim 1, wherein said tip is made of a sapphire.

11. Instrument as claimed in claim 1, wherein said operating end of the tip is configured to emit a highly divergent laser beam which acts on the tissues by contact of said operating end on said tissues.

12. A surgical device comprising:
a plurality of laser sources and a surgical instrument comprising a handpiece, associated with which is a tip forming a waveguide and terminating with an operating end, designed to act on the tissues and from which a laser radiation is emitted and which receives said laser radiation from a fiber optic device wherein said tip is designed to collect the laser emission coming from a plurality of optical fibers couplable with said tip at a coupling face and to convey said laser emissions towards said operating end;
a plurality of optical fibers, associated with said laser sources, connecting said laser sources to the tip of said instrument.

13. A device as claimed in claim 12, wherein said laser sources emit at least two different wavelengths, the emissions at said different wavelengths being combined in the tip of said instrument.

14. Device as claimed in claim 13, wherein a first wavelength is selected to obtain a cutting effect of the tissues and a second wavelength is selected to obtain a hemostatic effect of the vessels severed by the first wavelength.

15. Instrument as claimed in claim 2, wherein said tip has an input portion, between said face for coupling with the optical fibers and said operating end, with a substantially constant cross section.

16. Instrument as claimed in claim 3, wherein said tip has an input portion, between said face for coupling with the optical fibers and said operating end, with a substantially constant cross section.

17. Instrument as claimed in claim 2, wherein said tip has a substantially cylindrical input portion.
